(19) United States
(12) Patent Application Publication
(10) Pub. No.: US 2025/0034123 A1
(43) Pub. Date: Jan. 30, 2025

(54) A SYNTHESIS METHOD OF BENZOXAZOLE BASED OPTICAL BRIGHTENERS

(71) Applicant: AKDENIZ CHEMSON KIMYA SANAYI VE TICARET ANONIM SIRKETI, Caddesi/Izmir (TR)

(72) Inventors: Mehmet BASALP, Bornova/Izmir (TR); Gokay GUREL, Izmir (TR)

(21) Appl. No.: 18/717,185

(22) PCT Filed: Dec. 28, 2021
(86) PCT No.: PCT/TR2021/051541

§ 371 Date: Jun. 6, 2024

(51) Int. Cl. C07D 413/14; C07D 263/64

(52) U.S. Cl. CPC C07D 413/14; C07D 263/64

(57) ABSTRACT

The present invention is a synthesis method for benzoxazole based optical brighteners characterized by comprising the steps of; placing in a reactor vessel at least one acid selected from thiophene-2,5-dicarboxylic acid or stilbene-4,4′-dicarboxylic acid, at least one catalyst and at least one compound selected from liquid paraffin and/or xylene; obtaining a mixture by adding 2-amino-4-tert-butylphenol in the reactor vessel if the said at least one acid is selected as thiophene-2,5-dicarboxylic acid or adding 2-aminophenol in the reactor vessel if the said at least one acid is selected as stilbene-4,4′-dicarboxylic acid; mixing the obtained mixture for a first time period under nitrogen atmosphere to carry out the synthesis reaction of benzoxazole based optical brighteners; during mixing the obtained mixture, increasing the temperature of the said mixture gradually to a first temperature; after mixing obtained mixture for the first time period, filtering the mixture; after filtering the mixture, obtaining a first benzoxazole based optical brightener or a second benzoxazole based optical brightener.

### TECHNICAL FIELD

The present invention relates to a synthesis method of benzoxazole based optical brighteners for polymer applications.

### BACKGROUND OF THE INVENTION

Optical brighteners, also called as fluorescent whitening agents or optical whitening agents, are chemical compounds that absorb light in the ultraviolet and violet region of the electromagnetic spectrum, and re-emit light in the blue region by fluorescence. The aforementioned optical brighteners have been used for many years to improve the colour of various polymers. They mask inherent yellowness in polymers and make polymers appear whiter. The most commonly used optical brighteners are bis-benzoxazolyl-stilbene and bis-benzoxazolyl-thiophene.

Within the known state of art, an example which is related to the process for the preparation of bis-benzazolyl compounds used as optical whitening agents is disclosed in EP1326850B1. In the said patent document, the synthesis reaction of the optical whitening agents takes place in the presence of an acidic catalyst and optionally in the presence of a secondary solvent capable of removing water from the reaction mixture. At the end of the synthesis reaction, the mentioned optical whitening agents are dissolved in the solvents. However, these agents have to be removed from the solvents and they have to be purified.

Another U.S. Pat. No. 3,407,196A discloses a reaction between dicarboxylic acid chloride and ortho-aminophenol in the presence of solvents such as dichlorobenzene.

In addition, in the patent application No. CH439292A, a process for the preparation of new derivatives of thiophene is disclosed. In the mentioned process, the reaction occurs by melting dicarboxylic acid and orthoaminophenols. However, during said process, the temperature of reaction has to be risen to uncontrolled high temperatures.

On the other hand, the synthesis methods disclosed in mentioned documents possess solvents which have high boiling points or chlorinated solvents. In order to remove these solvents from the reaction mixture, the synthesis methods disclosed in the said documents have additional purification steps. Because of mentioned additional purification steps, the synthesis methods disclosed in the said patent documents take a long time and also the yield of optical brighteners obtained in the reaction is too low. Moreover, the synthesis methods disclosed in the said patent documents cause environmental problems.

### BRIEF DESCRIPTION OF THE INVENTION

The present invention provides a synthesis method of benzoxazole based optical brighteners. The synthesis method of benzoxazole based optical brighteners comprises the steps of:
placing in a reactor vessel at least one acid selected from thiophene-2,5-dicarboxylic acid or stilbene-4,4′-dicarboxylic acid, at least one catalyst and at least one compound selected from liquid paraffin and/or xylene;
obtaining a mixture by adding 2-amino-4-tert-butylphenol in the reactor vessel if the said at least one acid is selected as thiophene-2,5-dicarboxylic acid or adding 2-aminophenol in the reactor vessel if the said at least one acid is selected as stilbene-4,4′-dicarboxylic acid;
mixing the obtained mixture for a first time period under nitrogen atmosphere to carry out the synthesis reaction of benzoxazole based optical brighteners;
during mixing the obtained mixture, increasing the temperature of the said mixture gradually to a first temperature;
after mixing obtained mixture for the first time period, filtering the mixture;
after filtering the mixture, obtaining a first benzoxazole based optical brightener or a second benzoxazole based optical brightener.

Thanks to the synthesis method provided by the present invention, highly efficient benzoxazole based optical brighteners are obtained. Since the said synthesis method provided by the present invention does not have additional purification steps, the said method is environmental friendly and low-cost. Moreover, due the addition of liquid paraffin in the said synthesis method, the synthesis reaction of benzoxazole based optical brighteners takes place in short time period.

### OBJECT OF THE INVENTION

An object of the present invention is to provide a synthesis method of benzoxazole based optical brighteners.

Another object of the present invention is to provide a synthesis method of benzoxazole based optical brighteners for polymer applications.

Another object of the present invention is to provide a low-cost and environmental friendly synthesis method of benzoxazole based optical brighteners.

Another object of the present invention is to provide a highly efficient synthesis method of benzoxazole based optical brighteners.

### DESCRIPTION OF THE INVENTION

Optical brighteners, also known as fluorescent whitening agents or optical whitening agents, absorb light in the ultraviolet and violet region and emit visible blue lights. The mentioned optical brighteners are chemical compounds that have a system of conjugated double bonds and electron-donating groups to show the high fluorescence activity. The aforementioned optical brighteners are often used to enhance the colour of various polymers. Optical brighteners make polymers appear whiter by masking inherent yellowness in polymers. Bis-benzoxazolyl-stilbene and bis-benzoxazolyl-thiophene are most commonly used as optical brighteners. In the state of art, various processes for the preparation of these optical brighteners are improved. However, the aforementioned synthesis methods possess solvents which have high boiling points or chlorinated solvents. To remove these solvents from the reaction mixture in the synthesis methods known in the art, the synthesis methods have additional purification steps. Due to the said additional steps, the synthesis methods take too much time and also have low yield of optical brighteners. Furthermore, the mentioned additional purification steps in the synthesis methods cause environmental problems. Therefore, in the present invention, a synthesis method of benzoxazole based optical brighteners for solving the above mentioned-problems is provided.

According to the present invention, the synthesis method of benzoxazole based optical brighteners comprises the steps of:
placing in a reactor vessel at least one acid selected from thiophene-2,5-dicarboxylic acid or stilbene-4,4′-dicarboxylic acid, at least one catalyst and at least one compound selected from liquid paraffin and/or xylene;
obtaining a mixture by adding 2-amino-4-tert-butylphenol in the reactor vessel if the said at least one acid is selected as thiophene-2,5-dicarboxylic acid or adding 2-aminophenol in the reactor vessel if the said at least one acid is selected as stilbene-4,4′-dicarboxylic acid;
mixing the obtained mixture for a first time period under nitrogen atmosphere to carry out the synthesis reaction of benzoxazole based optical brighteners;
during mixing the obtained mixture, increasing the temperature of the said mixture gradually to a first temperature;
after mixing obtained mixture for the first time period, filtering the mixture;
after filtering the mixture, obtaining a first benzoxazole based optical brightener or a second benzoxazole based optical brightener.

In an exemplary embodiment of the invention, if the said at least one acid is selected as thiophene-2,5-dicarboxylic acid, 2-amino-4-tert-butylphenol is added into a reactor vessel in order to obtain a mixture. In the synthesis method provided by present invention, thiophene-2,5-dicarboxylic acid, 2-amino-4-tert-butylphenol, at least one catalyst (selected from metal based catalysts, Lewis acids and/or boric acid) and at least one compound selected from liquid paraffin and/or xylene are mixed preferably in a mechanical mixer, for a first time period (preferably 2-6 hours) under nitrogen atmosphere. During mixing the obtained mixture, the temperature of the obtained mixture is gradually increased to a first temperature (preferably 220-270° C.). After mixing obtained mixture for the first time period, the obtained mixture is filtered and a first benzoxazole based optical brightener is obtained. The said first benzoxazole based optical brightener is 2,5-thiophenediylbis(5-tert-butyl-1,3-benzoxazole).

The synthesis reaction mechanism of first benzoxazole based optical brightener is given as below:

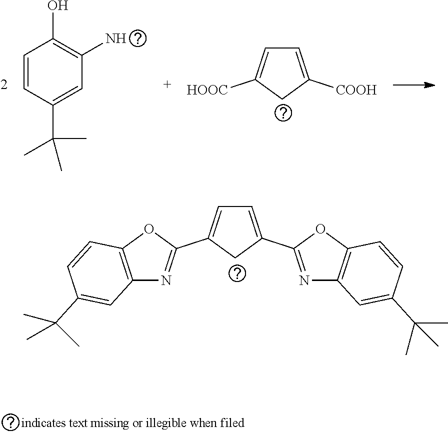 [SMILES: CC(C)(C)c1ccc(O)c(N)c1 ; O=C(O)C1=CC=C(C(=O)O)C1 ; CC(C)(C)c1ccc2oc(C3=CC=C(c4nc5cc(C(C)(C)C)ccc5o4)C3)nc2c1]

In an preferred embodiment of the invention, if the said at least one acid is selected as stilbene-4,4′-dicarboxylic acid, 2-aminophenol is added into a reactor vessel in order to obtain a mixture. In the synthesis method provided by present invention, stilbene-4,4′-dicarboxylic acid, 2-aminophenol, at least one catalyst (selected from metal based catalysts, Lewis acids and/or boric acid) and at least one compound selected from liquid paraffin and/or xylene are mixed preferably in a mechanical mixer, for a first time period (preferably 2-6 hours) under nitrogen atmosphere. During mixing the obtained mixture, the temperature of the obtained mixture is gradually increased to a first temperature (preferably 220-270° C.). After mixing obtained mixture for the first time period, the obtained mixture is filtered and a second benzoxazole based optical brightener is obtained. The said second benzoxazole based optical brightener is 4,4′-Bis(2-benzoxazolyl)stilbene.

The reaction mechanism of synthesis of second benzoxazole based optical brightener is given as below:

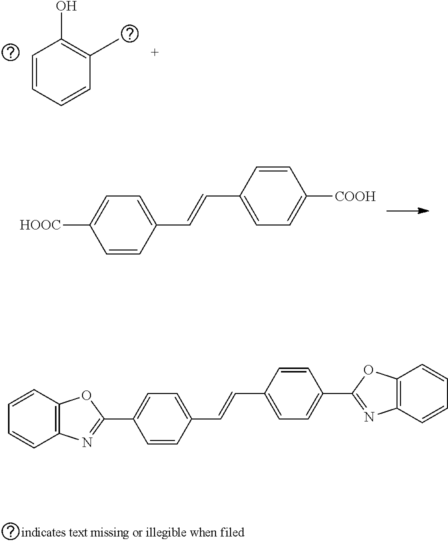

In an exemplary embodiment of the invention, the said liquid paraffin is utilized as nonpolar disintegrant. Due to its nonpolar feature, it can be easily removed from the first or second benzoxazole based optical brightener during the filtration of the mixture. In addition, after removing liquid paraffin from the first or second benzoxazole based optical brightener, liquid paraffin can be used again as disintegrant. Moreover, thanks to liquid paraffin being able to reach high temperatures, the benzoxazole based optical brighteners can be obtained in short time period (preferably 2-6 hours).

In a preferred embodiment of the invention, the said first or second benzoxazole based optical brighteners obtained by present invention are used for polymer applications. The mentioned benzoxazole based optical brighteners cover the yellowness of the polymers and make them appear whiter.

Thanks to the synthesis method provided by the present invention, highly efficient benzoxazole based optical brighteners are obtained. Since the said synthesis method provided by the present invention does not have additional purification steps, the said method is environmental friendly and low-cost. Additionally, thanks to the addition of liquid paraffin utilized as nonpolar disintegrant in the said synthesis method, the temperature of the obtained mixture reaches the high temperatures and the synthesis reaction takes place in short time period.

1. A synthesis method for benzoxazole based optical brighteners, comprising the steps of:
placing in a reactor vessel at least one acid selected from thiophene-2, 5-dicarboxylic acid or stilbene-4,4′-dicarboxylic acid, at least one catalyst and liquid paraffin;
obtaining a mixture by adding 2-amino-4-tert-butylphenol in the reactor vessel when the said at least one acid is selected as thiophene-2, 5-dicarboxylic acid or adding 2-aminophenol in the reactor vessel when the said at least one acid is selected as stilbene-4,4′-dicarboxylic acid;
mixing the obtained mixture for a first time period under nitrogen atmosphere to carry out the synthesis reaction of benzoxazole based optical brighteners;
during mixing the obtained mixture, increasing the temperature of the said mixture gradually to a first temperature;
after mixing obtained mixture for the first time period, filtering the mixture; and
after filtering the mixture, obtaining a first benzoxazole based optical brightener or a second benzoxazole based optical brightener.

2. The synthesis method for benzoxazole optical brighteners according to claim 1, wherein the first time period is within a range of 2-6 hours.

3. The synthesis method for benzoxazole optical brighteners according to claim 1, wherein the first temperature is between 220 to 270° C.

4. The synthesis method for benzoxazole optical brighteners according to claim 1, wherein the first benzoxazole based optical brightener is 2,5-thiophenediylbis(5-tert-butyl-1,3-benzoxazole).

5. The synthesis method for benzoxazole optical brighteners according to claim 1, wherein the second benzoxazole based optical brightener is 4,4′-Bis(2-benzoxazolyl)stilbene.

6. The synthesis method for benzoxazole optical brighteners according to claim 1, wherein the at least one catalyst is selected from metal based catalysts, Lewis acids and/or boric acid.
